(19) United States
(12) Patent Application Publication
(10) Pub. No.: US 2022/0142630 A1
(43) Pub. Date: May 12, 2022

(54) APPLICATOR FOR A TWO-COMPONENT FABRIC ADHESIVE

(71) Applicant: ADHESYS MEDICAL GMBH, Aachen (DE)

(72) Inventors: Hayley A. LOUGH, Boston, MA (US); Matthew BAYES, Boston, MA (US); Ralf DORSCHEID, Aachen (DE)

(21) Appl. No.: 17/431,949

(22) PCT Filed: Feb. 17, 2020
(86) PCT No.: PCT/EP2020/054083

§ 371 Date: Aug. 18, 2021

(30) Foreign Application Priority Data
Feb. 20, 2019 (EP) 19158345.9

(51) Int. Cl. A61B 17/00; A61L 24/04

(52) U.S. Cl. CPC A61B 17/00491; A61L 2430/34; A61B 2017/00495; A61L 24/046

(57) ABSTRACT

The present invention relates to a dispensing system (1) having at least two chambers (2,3) for a tissue adhesive system comprising at least two components, wherein first chamber (2) is designed as a hollow cylinder (H), which has a closed design on an end face (2a) and an open design on its opposite end face (2b) and second chamber (3) in a solid cylinder (V) is designed as an annular gap (4) and annular gap (4) extends from an end face (3a) facing hollow cylinder (H) to a mixing chamber (5) arranged on opposite end (3b) of solid cylinder (V) and hollow cylinder (H) is arranged on the side of its open end face (2b) in annular gap (4) of solid cylinder (V) so it can move in longitudinal direction (L), wherein at least one feed duct (6) is designed in solid cylinder (V), which extends on the inside of annular gap (4) from end face (3a) of solid cylinder (V) facing hollow cylinder (H) to mixing chamber (5), wherein a mixing tube (7) is provisioned in mixing chamber (5), which projects laterally from solid cylinder (V) and is arranged so it can move essentially transversely to the longitudinal expansion of solid cylinder (V) and closes the inside of mixing tube (7) off from feed duct (6) and annular gap (4) in a first position and unblocks it in a second position. The invention also relates to a kit consisting of a dispensing system, which is filled with tissue adhesive system comprising at least two components as well as a method for closing a wound using said kit.

The present invention relates to a dispensing system having at least two chambers for a tissue adhesive system comprising at least two components. The invention also relates to a kit consisting of a dispensing system, which is filled with a tissue adhesive system comprising at least two components, as well as a method for closing a wound using such a kit.

Two-component tissue adhesive systems (2-component tissue adhesive systems) as well as relevant dispensing systems having two chambers are known from the prior art. EP 2 173 782 B1 discloses a 2-chamber dispensing system, which is filled with a 2-component tissue adhesive system, of which the one component is an isocyanate-functional prepolymer consisting of a polyisocyanate and a polyol and the second component is an amino-functional aspartic acid ester. Such 2-chamber dispensing systems are commonly designed as a medical syringe having two cylinders arranged parallel to each other that are synchronously squeezed, wherein the two components can be combined at the end of the syringe in one pipe and applied to the desired location using an applicator equipped with a static mixer.

The fact that this solution can only be operated by qualified personnel may be perceived as a disadvantage. Both hands are typically required for this as well. In addition, these syringe-type 2-chamber dispensing systems with cylinders arranged parallel to each other are not particularly mechanically stable and, for example, are susceptible to bending, which is facilitated in particular by the design-related elongated and concurrently thin shape as the plungers of the syringe are normally fully extracted due to the filled syringe cylinders.

Thus, the object of the present invention was to provide a dispensing system having at least two chambers for a tissue adhesive system comprising at least two components capable of being operated by non-medically trained persons as well, preferably with one hand so that injuries at one's own arm or hand can likewise be treated. The dispensing system should preferably be designed mechanically more stable than the previously known syringe-type 2-chamber dispensing systems.

This object is achieved pursuant to the invention through a dispensing system having at least two chambers for a tissue adhesive system comprising at least two components, wherein the first chamber is designed as a hollow cylinder, which has a closed design on an end face and an open design on its opposite end face and

the second chamber in a solid cylinder is designed as an annular gap and the annular gap extends from an end face facing the hollow cylinder to a mixing chamber arranged on the opposite end of the solid cylinder and the hollow cylinder is arranged on the side of its open end face in the annular gap of the solid cylinder so it can move in the longitudinal direction, wherein at least one feed duct is designed in the solid cylinder, which extends on the inside of the annular gap from the end face of the solid cylinder facing the hollow cylinder to the mixing chamber, wherein a mixing tube is provisioned in the mixing chamber, which projects laterally from the solid cylinder and is arranged so it can move essentially transversely to the longitudinal expansion of the solid cylinder and which closes the inside of the mixing tube off from the feed duct and the annular gap in a first position and unblocks it in a second position.

The invention is based on the finding that the dispensing system can be designed substantially more compactly than previously known syringe-type 2-chamber dispensing systems due to the arrangement and design of the chambers enabling them to move inside each other. As a result, the dispensing system can have, for example, a rectangular geometry, which is significantly more rigid than a filled 2-chamber syringe. This also enables the dispensing system to be designed more ergonomically to the hand, such that it can also be compressed in one hand by persons having little or no practice using it and in this way, the 2-component tissue adhesive system can be pressed out.

The dispensing system pursuant to the invention is preferably designed with two chambers for a tissue adhesive system comprising two components.

The dispensing system pursuant to the invention also has a hollow cylinder and a solid cylinder. In the context of the present invention, a cylinder does not merely denote a simple circular cylinder, but also cylinders with an oval, polygonal/angular, e.g. square, hexagonal or prismatic cross-sectional area, ellipsoid or trapezoid cross-sectional area. Here, the hollow cylinder and the solid cylinder are synchronized in such a manner that they can be moved inside each other. The longitudinal expansion of the hollow cylinder is preferably selected such that the height of the inner wall is substantially identical to the depth of the annular gap.

The solid cylinder can be constructed monolithically, for example from a solid material, or have hollow spaces as well for saving material, which however do not serve for filling with components of the 2-component tissue adhesive system.

The solid cylinder is preferably constructed from two elements, which simplifies fitting it together with the hollow cylinder. Here, the first element essentially accommodates the annular gap while the second element can be slid over the first element and, for example, firmly attached to the first element by means of a latch connection, such that the hollow cylinder and the solid cylinder form one unit. The solid cylinder may have hollow spaces on the inside which are closed towards the adhesive components to save material. Thus, these hollow spaces will not become filled with adhesive components when filling the annular gap of the solid cylinder.

The hollow cylinder and the solid cylinder are preferably manufactured from a rigid material, particularly of a plastic material, the polymer matrix of which is preferably selected from polyolefins, preferably polyethylene and polypropylene, ABS plastic, polycarbonate, polyester, preferably polyethylene terephthalate, poly(methyl)methacrylate, polyvinyl chloride, (2-component) epoxy resins, polystyrene, as well as mixtures and mixed polymerisates thereof. The hollow cylinder and the solid cylinder can be manufactured using any of the methods known to specialists for molding the aforementioned materials, such as injection molding.

With the dispensing system pursuant to the invention, an end stop device can be designed in the area of the impact edge of the open end face of the hollow cylinder, which interacts with a corresponding end stop device designed on the end face of the solid cylinder facing the hollow cylinder in such a way that the hollow cylinder and the solid cylinder can only be pulled apart to the point the end stop devices touch each other. This ensures that the chambers are only filled until the end stop is reached when filling the dispensing system. The end stop device also prevents the dispensing system from being unintentionally pulled apart.

The hollow cylinder and the solid cylinder can also be equipped with a securing device, which secures the hollow cylinder and the solid cylinder from being unintentionally pushed against each other. For this, a plastic tab can be provisioned on one of the two cylinders, which corresponds to a respective recess on the other cylinder and which breaks off in the event of intentional use due to the increased application of force thus releasing the cylinders so they can be pushed against each other.

The end stop device on the hollow cylinder is expediently designed as a circumferential annular bead, which interacts with a mating surface surrounding the inside on the solid cylinder as a corresponding end stop device. Here, a circumferential sealing element can also be provisioned on the annular bead, which interacts with at least one, preferably with both, longitudinal surfaces of the annular gap providing a seal.

The volumes of both chambers can be freely chosen over broad ranges and are expediently coordinated in such a way that they have the same ratio as that, at which the components of the tissue adhesive system comprising two components must be mixed together. The volume ratio of the chambers is preferably 1:10 to 10:1, preferably 5:1 to 1:5.

According to a preferred embodiment of the dispensing system pursuant to the invention, the volume of the first chamber is greater than that of the second chamber, wherein the volume ratio of the first chamber to the second chamber is 5:1 to 3:1, preferably 4.5:1 to 3.5:1.

As already specified above, the hollow cylinder and the solid cylinder can have a circular, oval, polygonal/angular, ellipsoid or trapezoid cross-sectional surface, particularly a rectangular cross-sectional surface with rounded edges, wherein the ratio of the long side to the short side is preferably at least 3:1, more preferably at least 4:1.

To enhance the handling and grip of the dispensing system pursuant to the invention, the outside of the hollow cylinder and/or the solid cylinder can be equipped with a profiling, particularly a ribbed profile, running in longitudinal direction.

In a preferred further embodiment of the dispensing system pursuant to the invention, mixing elements are arranged within the mixing tube, wherein the mixing tube is designed in particular as a static mixer. This can ensure that the reactants of the 2-component or multi-component tissue adhesive system are adequately mixed by the time they leave the mixing tube. A latching mechanism can also be provisioned on the mixing tube, which secures the mixing tube in the first position so it cannot be unintentionally pulled out. A retraction locking device can be provisioned on the mixing tube, which is designed in such a way that it secures the mixing tube after being moved to the second position and prevents it from moving back, wherein the retraction locking device is preferably designed, for example, as a latching element (wing or barb). The wing, for example, may consist of a mechanically pretensioned plastic film section, which stops after the mixing tube is pulled out due to its pretensioning from the mixing tube and thus blocks it from being pushed back. The mixing tube can also be equipped with a removable plug on its side projecting out of the solid cylinder.

With the dispensing system pursuant to the invention, it is also possible to provision that the mixing tube is guided in the mixing chamber so that it cannot twist and is equipped with one or more openings on its inward facing end on the side facing the feed duct, through which the feed duct and/or the annular gap can be connected to the inside of the mixing tube if the mixing tube is put in the second position. For this purpose, a keyway or guide spring can be provisioned in the mixing chamber, which corresponds to a respective guide groove on the mixing tube. The opposite arrangement of keyway or guide spring and guide groove is possible.

In a preferred further embodiment of the dispensing system pursuant to the invention, the mixing chamber is designed within the solid cylinder. This is beneficial as in this way no additional component is required, which simplifies the manufacturing process and also reduces the risk of leaks. The mixing chamber essentially serves to receive and to guide the mixing tube, whereas the actual mixing of the components occurs inside the mixing tube.

It is particularly preferred that an annular gap seal is provisioned in the mixing chamber between the junction of the annular gap and the exterior opening of the mixing chamber, which serves to seal between the mixing chamber and the outer surface of the mixing tube in position 1 and position 2. This prevents the contents of the chambers from being able to leak out past the mixing tube. Regardless of this, additional annular gap seals can be provisioned further inside, for example, one between the outlet of the annular gap and the feed duct, so as to prevent unintentionally mixing of the at least two components outside of the mixing tube in the mixing chamber. Another annular gap can be arranged on the side of the feed duct facing away from the outlet of the annular gap. This can inhibit the component contained in the hollow cylinder from leaking into the mixing chamber.

The feed duct runs expediently essentially parallel to the annular gap and is spaced particularly at least 2 mm from it, preferably at least 3 mm.

For a better seal and protection particularly against light, oxygen and moisture penetration, the hollow cylinder and the solid cylinder can be collectively enclosed in a foil bag, wherein the foil bag is preferably provided with a tear line in the area of the projecting mixing tube, which enables a systematic opening of the foil bag on the mixing tube. The foil bag is distinguished essentially by the fact that it consists of a material or a material composition, which is impermeable to light and/or moisture and also ageing-resistant and it ensures a long-term sterile barrier. For example, the foil bag may consist of a composite film, such as aluminum foil, for example, with a polyester film (PET) and/or a film consisting of polyethylene (PE), which is laminated with a polymer film on one or both sides. The foil bag can also be vacuumed or filled with an inert shielding gas as well, such as nitrogen.

Another object of the present invention relates to a kit consisting of a dispensing system pursuant to the invention and a tissue adhesive system comprising two components located therein, as well as optionally a process manual for wound closure using the kit, wherein the tissue adhesive system comprises preferably at least one aspartic acid ester as a hardener with optionally at least one filler as the first component and an isocyanate-functional prepolymer as a second component, wherein the first component is particularly preferably located in the first chamber and the second component is preferably located in the second chamber of the dispensing system. As a 2-component tissue adhesive system, those disclosed in EP 2 173 782 B1, EP 2 182 018 B1, WO 2011/006606 A1, WO 2010/006714 A2, A3, EP 2 699 615 B1, WO 2013/104563 A1, WO 2012/107375 A1, WO 2013/092506 A1, EP 2 699 614 B1 or WO 2013/092504 A1 are particularly preferably used.

The present invention also relates to a method for wound closure using a kit pursuant to the invention comprising the steps:
a) Pulling out the mixing tube until the second position is reached;
b) Exerting pressure on the hollow cylinder and the solid cylinder relative to each other in the longitudinal direction of the cylinders, whereby both components of the tissue adhesive system are conveyed in the direction of the mixing chamber and mixed together upon flowing through the mixing tube;
c) Optional discarding at least the first 10 mm of the tissue adhesive discharging out of the mixing tube;
d) Applying the tissue adhesive discharged through the continued application of pressure from mixing tube 7 onto the wound;
e) Allowing the tissue adhesive to cure.

If the system is enclosed in a foil bag, it can be tom open in the area of the mixing tube and the mixing tube can be pulled out, followed by the other specified process steps. Thus, it is not necessary here to remove the foil bag.

The invention relates particularly to the following embodiments:
1. A dispensing system 1 having at least two chambers 2, 3 for a tissue adhesive system comprising at least two components, wherein first chamber 2 is designed as a hollow cylinder H, which has a closed design on an end face 2a and an open design on its opposite end face 2b and second chamber 3 in a solid cylinder V is designed as an annular gap 4 and annular gap 4 extends from an end face 3a facing hollow cylinder H to a mixing chamber 5 arranged on an opposite end 3b of solid cylinder V and hollow cylinder H is arranged on the side of its open end face 2b in annular gap 4 of solid cylinder V so it can move in longitudinal direction L,

wherein at least one feed duct 6 is designed in solid cylinder V, which extends on the inside of annular gap 4 from end face 3a of solid cylinder V facing hollow cylinder H to mixing chamber 5, wherein a mixing tube 7 is provisioned in mixing chamber 5, which projects laterally from solid cylinder V and is arranged so it can move essentially transversely to the longitudinal expansion of solid cylinder V and which closes the inside of mixing tube 7 off from opposite feed duct 6 and annular gap 4 in a first position and unblocks it in a second position.
2. Dispensing system according to embodiment 1, wherein hollow cylinder H und solid cylinder V are manufactured from a rigid material, particularly of a plastic material, the polymer matrix of which is preferably selected from polyolefins, preferably polyethylene and polypropylene, ABS plastic, polycarbonate, polyester, preferably polyethylene terephthalate, poly methyl methacrylate, polyvinyl chloride, epoxy resins, polystyrene, as well as mixtures and mixed polymerisates thereof.
3. Dispensing system according to embodiment 1 or 2, wherein an end stop device 8 is designed in the area of the impact edge of open end face 2b of hollow cylinder H, which interacts with a corresponding end stop device 9 designed on end face 3a of solid cylinder V facing hollow cylinder H in such a way that hollow cylinder H and solid cylinder V can only be pulled apart to the point end stop devices 8, 9 touch each other.
4. Dispensing system according to embodiment 3, wherein end stop device 8 on hollow cylinder H is designed as a circumferential annular bead, which interacts with a mating surface surrounding the inside on solid cylinder V as a corresponding end stop device 9.
5. Dispensing system according to embodiment 4, wherein a circumferential sealing element 11 is provisioned on the annular bead, which interacts with at least one, preferably with both, longitudinal surfaces of annular gap 4 providing a seal.
6. Dispensing system according to one of the previous embodiments, wherein the longitudinal expansion of hollow cylinder H is preferably selected such that the height of the inner wall is substantially identical to the depth of annular gap 4.
7. Dispensing system according to one of the previous embodiments, wherein the dispensing system has two chambers 2, 3 and the volume ratios of both chambers 2, 3 to each other are 1:10 to 10:1, preferably 5:1 to 1:5.
8. Dispensing system according to one of the previous embodiments, wherein the volume ratio of first chamber 2 to second chamber 3 is 5:1 to 3:1, preferably 4.5:1 to 3.5:1.
9. Dispensing system according to one of the previous embodiments, wherein hollow cylinder H and solid cylinder V are equipped with a securing device, which secures hollow cylinder H and solid cylinder V from being unintentionally pushed against each other.
10. Dispensing system according to one of the previous embodiments, wherein hollow cylinder H and solid cylinder V have a circular, oval, polygonal, ellipsoid, trapezoid cross-sectional surface, particularly a rectangular cross-sectional surface with rounded edges, wherein the ratio of the long side to the short side is preferably at least 3:1, more preferably at least 4:1.
11. Dispensing system according to one of the previous embodiments, wherein the outside of hollow cylinder H and/or solid cylinder V is equipped with a profiling 12, particularly a ribbed profile, running in longitudinal direction.
12. Dispensing system according to one of the previous embodiments, wherein mixing elements are arranged within mixing tube 7, wherein mixing tube 7 is designed in particular as a static mixer.
13. Dispensing system according to one of the previous embodiments, wherein a latching mechanism 12 is provisioned on mixing tube 7, which secures mixing tube 7 in the first position so it cannot be unintentionally pulled out.
14. Dispensing system according to one of the previous embodiments, wherein a retraction locking device 13 is provisioned on mixing tube 7, which is designed in such a way that it secures mixing tube 7 after being moved to the second position and prevents it from moving back, wherein retraction locking device 13 is preferably designed as a latching element, particularly as a wing or a barb.
15. Dispensing system according to one of the previous embodiments, wherein mixing tube 7 is guided in mixing chamber 5 so that it cannot twist and is equipped with one or more openings 14, 15 on its inward facing end on the side facing the feed duct, through which feed duct 6 and/or annular gap 4 can be connected to the inside of mixing tube 7 if mixing tube 7 is put in the second position.
16. Dispensing system according to one of the previous embodiments, wherein mixing tube 7 is equipped with a removable plug 16 on its side projecting out of solid cylinder V.
17. Dispensing system according to one of the previous embodiments, wherein mixing chamber 5 is designed within solid cylinder V.
18. Dispensing system according to one of the previous embodiments, wherein an annular gap seal 17 is provisioned in mixing chamber 5 between the junction of the annular gap and the exterior opening of the mixing chamber, which serves to seal between mixing chamber 5 and the outer surface of mixing tube 7 in position 1 and position 2.
19. Dispensing system according to one of the previous embodiments, wherein feed duct 6 essentially runs parallel to annular gap 4 and is spaced particularly at least 2 mm from it, preferably at least 3 mm.
20. Dispensing system according to one of the previous embodiments, wherein hollow cylinder H and solid cylinder V are collectively enclosed in a foil bag 21, wherein foil bag 21 is preferably provided with a tear line in the area of projecting mixing tube 7, which enables a systematic opening of foil bag 21 on mixing tube 7.
21. A kit 20 consisting of a dispensing system 1 according to one of embodiments 1 to 20, and a tissue adhesive system comprising two components located therein, as well as optionally a process manual for wound closure using said kit, wherein the tissue adhesive system comprises preferably at least one aspartic acid ester as a hardener with optionally at least one filler as the first component and an isocyanate-functional prepolymer as a second component, wherein the first component is particularly preferably located in first chamber (2) and the second component is preferably located in second chamber (3) of the dispensing system.
22. A method for closing a wound using a kit in accordance with embodiment 21 comprising the steps:
   a) Pulling out mixing tube 7 until the second position is reached;
   b) Exerting pressure on hollow cylinder H and solid cylinder V relatively to each other in the longitudinal direction of cylinders H, V, whereby both components of the tissue adhesive system are conveyed in the direction of mixing chamber 5 and mixed together upon flowing through mixing tube 7;
   c) Optionally discarding at least the first 10 mm of tissue adhesive discharging out of mixing tube 7;
   d) Applying the tissue adhesive discharged through the continued application of pressure from mixing tube 7 onto the wound;
   e) Allowing the tissue adhesive to cure.

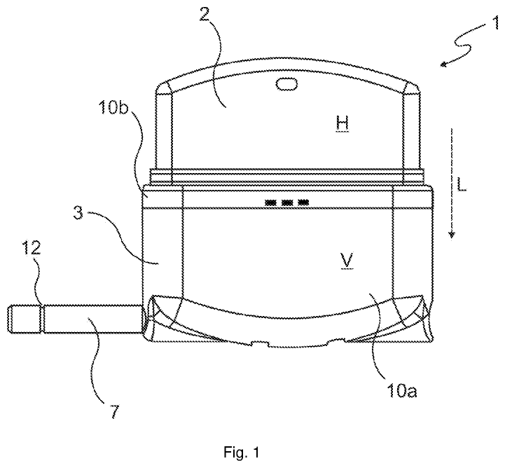

The present invention is clarified in greater detail below based on one of the design examples depicted in FIGS. 1 to 6. The following is presented therein:

FIG. 1 An embodiment of an apparatus pursuant to the invention in lateral plan view,

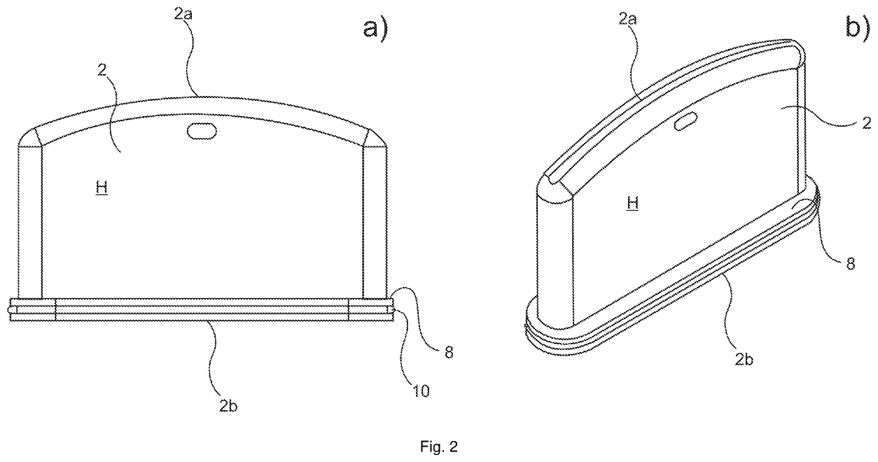

FIGS. 2a) and b) Detailed views of the first chamber of the apparatus from FIG. 1 diagonally from above,

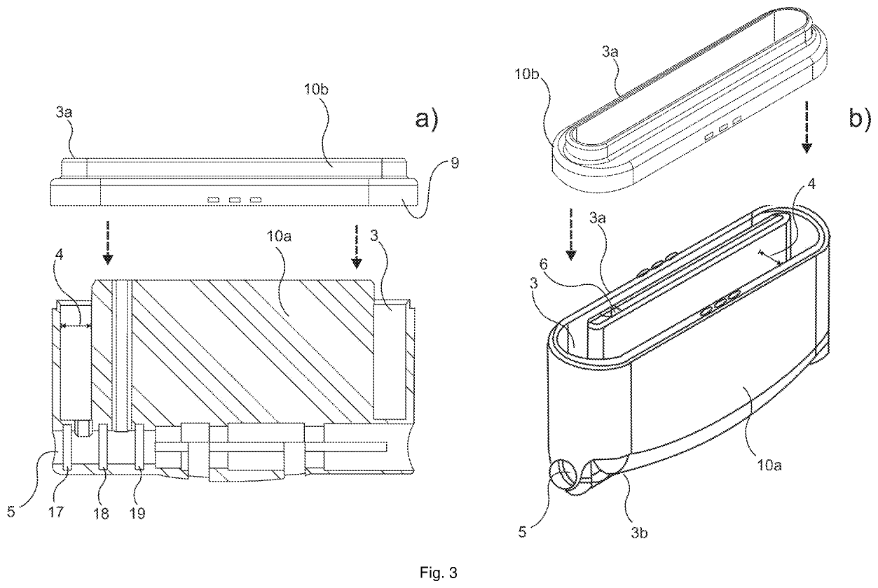

FIGS. 3a) and b) Detailed views of the second chamber of the apparatus from FIG. 1 diagonally from above,

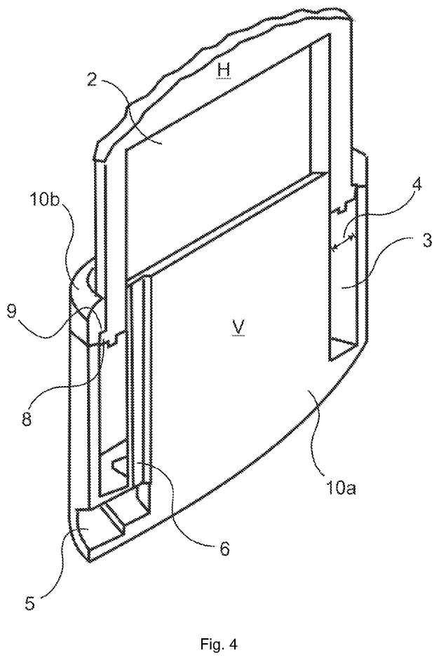

FIG. 4 A sectional view of the apparatus from FIG. 1 diagonally from above,

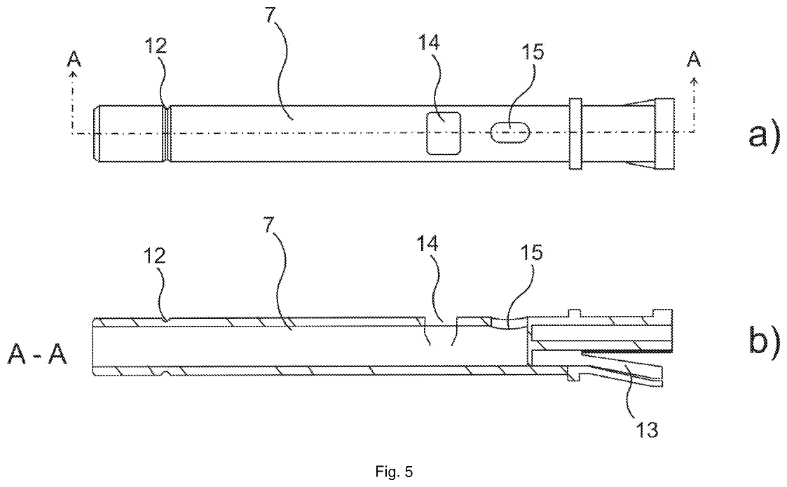

FIGS. 5a) and b) Detailed views of the mixing tube of the apparatus from FIG. 1 in lateral plan view and lateral sectional view, as well as

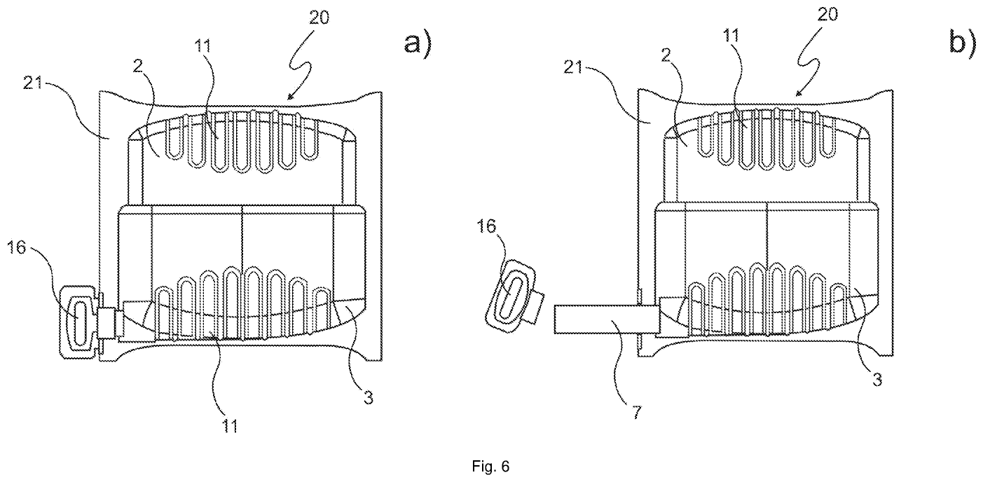

FIGS. 6a) and b) An embodiment of a kit pursuant to the invention prior to and after opening.

A dispensing system 1 pursuant to the invention is depicted in FIG. 1 in lateral plan view. Dispensing system 1 consists of two elongated, oval chambers 2, 3 for a tissue adhesive system comprising two components, for example, a system based on isocyanate-functional prepolymers on the basis of aliphatic polyisocyanates and amino-functional aspartic acid esters as one component and a polyol as a second component. Hollow cylinder H and solid cylinder V are manufactured from a rigid material, in the present case from polypropylene.

First chamber 2 is designed as a hollow cylinder H, which has a closed design on an end face 2a and an open design on its opposite end face 2b, thus forming a cavity for receiving a component of the tissue adhesive system. First chamber 2 is once again separately depicted in FIG. 2a) and b).

Second chamber 3 is designed as an oval annular gap 4 in a solid cylinder V. The second component of the tissue adhesive system can be filled in annular gap 4. Annular gap 4 extends from an end face 3a facing hollow cylinder H to a mixing chamber 5 designed therein on an opposite end 3b of solid cylinder V, wherein hollow cylinder H is arranged on the side of its open end face 2b in annular gap 4 of solid cylinder V so it can move in longitudinal direction L.

A mixing tube 7 is provisioned in mixing chamber 5, which is designed as a static mixer. Mixing tube 7 projects laterally from the solid cylinder V and is arranged essentially transversely to the longitudinal expansion of solid cylinder V.

The volumes of both chambers 2, 3 can be respectively adapted to the mixing ratio of the adhesive system, wherein the ratio of first chamber 2 to second chamber 3 at hand is approximately 3:1.

As seen in FIGS. 3a) and b), second chamber 3 consists of two elements 10a, 10b, which can be connected to each other firmly and gas-tight by means of a latching mechanism after being assembled in direction indicated by the dashed arrows. For the purpose of sealing between two elements 10a, 10b, one of these is equipped with a circumferential seal.

The division of second chamber 3 in two elements 10a, 10b serves the purpose of simplifying filling chambers 2, 3 with the components of the tissue adhesive system. Thus, initially first chamber 2 can be filled with the one component of the tissue adhesive system and assembled together with element 10a bearing annular gap 4 in such a way that the circumferential edge bead of first chamber 2 is inserted into annular gap 4. Oval, annular second element 10b is then slipped over first chamber 2 and connected to element 10a via the latching mechanism described above, whereby first chamber 2 is simultaneously clamped such that it can no longer become detached. Annular gap 4 of second chamber 3 is then filled with the other component of the tissue adhesive system. This occurs expediently prior to inserting mixing tube 7 through the opening of second chamber 3 provisioned for mixing tube 7. Alternatively, the base area of chamber 3 below mixing chamber 5 can also be designed to be removed, which simplifies filling into annular gap 4.

When filled and assembled, the bottom of the circumferential annular bead of first chamber 2 serves as a cylindrical surface and the top of the annular bead in conjunction with the circumferential bottom edge act as corresponding end stop devices 8, 9 of hollow cylinder H and solid cylinder V, such that hollow cylinder H and solid cylinder V can only be pulled apart to the point end stop devices 8, 9 touch each other. This prevents dispensing system 1 from being unintentionally opened. To ensure that the adhesive components are used as completely as possible when compressing the dispensing system, the longitudinal expansion of hollow cylinder H is selected such that the height of the inner wall is substantially identical to the depth of annular gap 4. This is particularly apparent from the sectional view in FIG. 4.

At least one feed duct 6 is designed in solid cylinder V, which extends on the inside of annular gap 4 from end face 3a of solid cylinder V facing hollow cylinder H to mixing chamber 5. The tissue adhesive component contained in hollow cylinder H is conveyed through feed duct 6 in the direction of mixing chamber 5 as soon as chambers 2, 3 are pushed into each other by compressing dispensing system 1. To ensure that no adhesive components can exit here on the interfaces between first and second chamber 2, 3, a circumferential sealing element 10 is provisioned externally on the annular bead, which interacts with both longitudinal surfaces of annular gap 4 providing a seal.

To prevent both adhesive components from prematurely and unintentionally mixing, mixing tube 7 closes the inside of mixing tube 7 off from feed duct 6 and annular gap 4 in a first position and only unblocks these openings in a second position, wherein the second position is defined by completely extracted mixing tube 7. This can be seen in particular in the detailed diagrams of mixing tube 7 in FIGS. 5a) and b). For this purpose, mixing tube 7 has two openings 14, 15 on its inward facing end on the side facing the feed duct 6, through which feed duct 6 and annular gap 4 can be connected to the inside of mixing tube 7 if mixing tube 7 is placed in the second position. To ensure that openings 14,15 remain in the proper position when extracting mixing tube 7, mixing tube 7 is guided in mixing chamber 5 so that it cannot twist.

FIGS. 5a) and b) further show that mixing tube 7 has a retraction locking device 13, which is designed such that it secures mixing tube 7 and prevents retraction after being moved into the second position, wherein retraction locking device 13 in the present case is designed as a latching element in the form of a barb, which stops therein after retracting mixing tube 7. A latching mechanism 12 in the form of an annular groove is embedded in the area of the front end of mixing tube 7, which secures mixing tube 7 from unintentionally slipping out when compressed with a corresponding pin on the outlet of mixing chamber 5.

Three annular gap seals 17, 18, 19 are provisioned in the mixing chamber 5 between the junction of annular gap 4 and the exterior opening of mixing chamber 5. Outer annular gap seal 17 serves as a seal between mixing chamber 5 and the outer surface of mixing tube 7 in position 1 and position 2. Middle annular gap seal 18 is arranged between the passage of annular gap 4 and feed duct 6 to prevent the two adhesive components from unintentionally mixing outside of mixing tube 7 in mixing chamber 5. Inside annular gap seal 19 is arranged on the side of feed duct 6 facing away from the passage of annular gap 4. Annular gap seal 19 can inhibit the component contained in hollow cylinder 2 from leaking into mixing chamber 5.

A kit 20 pursuant to the invention consisting of a dispensing system 1, as shown in FIGS. 1 to 5, is depicted in FIGS. 6a) and b). The respective exterior sides of hollow cylinder H and solid cylinder V are equipped with profiling 11 in the form of a ribbed profile running in the longitudinal direction, which enhances grip and stability. Two components of a polyurethane urea tissue adhesive system are filled in chambers 2, 3. The isocyanate functional prepolymer on the basis of aliphatic polyisocyanates and amino-functional aspartic acid esters is filled in annular gap 4 of the second chamber 3 and the polyol in the first chamber 2. For this purpose, as a 2-component tissue adhesive system, those presented in EP 2 173 782 B1, EP 2 182 018 B1, WO 2011/006606 A1, WO 2010/006714 A2, A3, EP 2 699 615 B1, WO 2013/104563 A1, WO 2012/107375 A1, WO 2013/092506 A1, EP 2 699 614 B1 or WO 2013/092504 A1 are used.

To ensure that no oxygen, no moisture or other environmental impacts can unfavorably alter the adhesive components, dispensing system 1 is shrink-wrapped in a foil bag 21 consisting of aluminized PET film and the outer end of mixing tube 7 is equipped with a removable plug 16. Removable plug 16 also serves to extract mixing tube 7 from mixing chamber 5 during use. For simplified handling, foil bag 21 is provided with a tear line in the area of projecting mixing tube 7, which enables a systematic opening of foil bag 21 on mixing tube 7. Thus, removing entire foil bag 21 is not required when using the system; rather it is sufficient to open it in the area of plug 16.

To fill kit 20 pursuant to the invention with a 2-component tissue adhesive system, it is possible to proceed in such a manner that first chamber 2 is filled with the one component of the tissue adhesive system in a dry inert gas atmosphere and assembled together with element 10a bearing annular gap 4 in such a way that the circumferential edge bead of first chamber 2 is inserted into annular gap 4. Oval, annular second element 10b is then stripped over first chamber 2 and connected to element 10a via the latching mechanism described above, whereby first chamber 2 is simultaneously clamped such that it can no longer become detached. Annular gap 4 of second chamber 3 is then filled with the other component of the tissue adhesive system. This occurs expediently prior to inserting mixing tube 7 through the opening of second chamber 3 provisioned for mixing tube 7. Alternatively, the base area of chamber 3 below mixing chamber 5 can also be designed to be removed, which simplifies filling into annular gap 4. Mixing tube 7 is then inserted into filled dispensing system 1 over latching mechanism 12 and shrink-wrapped under dry inert gas in a foil bag 21, the inside of which is aluminized. u

To use kit 20 pursuant to the invention for closing a wound, for example a laceration on the arm, by pulling on plug 16, foil bag 21 is simultaneously tom open, mixing tube 7 is pulled out until the end position is reached, and plug 16 is then removed by a slightly tilting pulling movement. This can also be done, for example, through the use of teeth if the arm injury does not enable the use of both hands. The dispensing system is then compressed with one hand so that first and second chamber 2, 3 are pushed together, whereby both components are conveyed into mixing tube 7, mixed there, and discharged at the opening of mixing tube 7. The first 2 cm of discharged compound can be discarded. The subsequent adhesive mixture strand is then applied directly to the bleeding wound, where the wound adhesive, accelerated through the moisture contact with the wound, hardens within 30 seconds to 3 minutes depending on the adhesive system, and thus stops the bleeding.

### LIST OF REFERENCE NUMBERS

1 Dispensing system
2 Chamber
2a End face
2b End face
3 Chamber
3a End face
3b End face
4 Annular gap
5 Mixing chamber
6 Feed duct
7 Mixing tube
8 End stop device
9 End stop device
10 Circumferential seal
10a Element
10b Element
11 Profiling
12 Latching mechanism
13 Retraction locking device
14 Opening
15 Opening
16 Plug
17 Outer annular gap seal
18 Middle annular gap seal
19 Inner annular gap seal
20 Kit
21 Foil bag
H Hollow cylinder
V Solid cylinder
L Longitudinal direction

1.-22. (canceled)

23. A dispensing system having at least two chambers for a tissue adhesive system comprising at least components,
wherein
first chamber is designed as a hollow cylinder, which has a closed design onan end face and an open design on its opposite end face and
second chamber in a solid cylinder is designed as an annular gap and annular gap extends from an end face facing hollow cylinder to a mixing chamber arranged on opposite end of solid cylinder and hollow cylinder is arranged on the side of its open end face in annular gap of solid cylinder so it can move in longitudinal direction,
wherein at least one feed duct is designed in solid cylinder, which extends on the inside of annular gap from end face of solid cylinder facing hollow cylinder to mixing chamber, wherein a mixing tube is provisioned in mixing chamber, which projects laterally from solid cylinder and is arranged so it can move essentially transversely to the longitudinal expansion of solid cylinder and closes the inside of mixing tube off from feed duct and annular gap in a first position and unblocks it in a second position.

24. Dispensing system according to claim 23, wherein hollow cylinder and solid cylinder are manufactured from a rigid material, particularly from a plastic material, the polymer matrix of which is preferably selected from polyolefins, preferably polyethylene and polypropylene, ABS plastic, polycarbonate, polyester, preferably polyethylene terephthalate, poly(methyl)methacrylate, polyvinyl chloride, (2-component) epoxy resins, polystyrene, as well as mixtures and mixed polymerisates thereof.

25. Dispensing system according to claim 23, wherein an end stop device is designed in the area of the impact edge of open end face of hollow cylinder, which interacts with a corresponding end stop device designed on end face of solid cylinder facing hollow cylinder in such a way that hollow cylinder and solid cylinder can only be pulled apart to the point end stop devices touch each other.

26. Dispensing system according to claim 25, wherein end stop device on hollow cylinder is designed as a circumferential annular bead, which interacts with a mating surface surrounding the inside on solid cylinder as a corresponding end stop device.

27. Dispensing system according to claim 26, wherein a circumferential sealing element is provisioned on the annular bead, which interacts with at least one longitudinal surfaces of annular gap providing a seal.

28. Dispensing system according to claim 23, wherein longitudinal expansion of hollow cylinder is selected such that the height of the inner wall is substantially identical to the depth of annular gap.

29. Dispensing system according to claim 23, wherein the dispensing system has two chambers and the volume ratio of chambers is from 1:10 to 10:1.

30. Dispensing system according to claim 23, wherein the volume ratio of first chamber to second chamber is from 5:1 to 3:1.

31. Dispensing system according to claim 23, wherein hollow cylinder and solid cylinder are equipped with a securing device, which secures hollow cylinder and solid cylinder from being unintentionally pushed against each other.

32. Dispensing system according to claim 23, wherein hollow cylinder and solid cylinder have a circular, oval, polygonal/angular, ellipsoid or trapezoid cross-sectional surface, particularly a rectangular cross-sectional surface with rounded edges, wherein the ratio of the long side to the short side is at least 3:1.

33. Dispensing system according to claim 23, wherein the outside of hollow cylinder and/or solid cylinder is equipped with a profiling running in the longitudinal direction.

34. Dispensing system according to claim 23, wherein mixing elements are arranged within mixing tube, wherein mixing tube is designed in particular as a static mixer.

35. Dispensing system according to claim 23, wherein a latching mechanism is provisioned on mixing tube, which secures mixing tube in the first position so it cannot be unintentionally pulled out.

36. Dispensing system according to claim 23, wherein a retraction locking device is provisioned on mixing tube, which is designed in such a way that it secures mixing tube after being moved to the second position and prevents it from moving back.

37. Dispensing system according to claim 23, wherein mixing tube is guided in mixing chamber so that it cannot twist and equipped with one or more openings on its inward facing end on the side facing the feed duct, through which feed duct and/or annular gap can be connected to the inside of mixing tube if mixing tube is put in the second position.

38. Dispensing system according to claim 23, wherein mixing tube is equipped with a removable plug on its side out of solid cylinder.

39. Dispensing system according to claim 23, wherein mixing chamber is designed within solid cylinder.

40. Dispensing system according to claim 23, wherein an annular gap seal is provisioned in mixing chamber between the junction of the annular gap and the exterior opening of the mixing chamber, which serves to seal between mixing chamber and the outer surface of mixing tube in position 1 and position 2.

41. Dispensing system according to claim 23, wherein feed duct runs essentially parallel to annular gap and is spaced from the annular gap at least 2 mm.

42. Dispensing system according to claim 23, wherein hollow cylinder and solid cylinder are collectively enclosed in a foil bag.

43. A kit consisting of a dispensing system according to claim 23, and a tissue adhesive system comprising two components, optionally a process manual for wound closure using said kit, wherein the tissue adhesive system comprises at least one aspartic acid ester as a hardener with optionally at least one filler as a first component, and an isocyanate-functional prepolymer as a second component, wherein the first component is located in first chamber and the second component is located in second chamber of the dispensing system.

44. A method for closing a wound using a kit in accordance with claim 43 comprising the steps:
a) pulling out mixing tube until the second position is reached;
b) exerting pressure on hollow cylinder and solid cylinder relatively to each other in the longitudinal direction of cylinders, whereby both components of the tissue adhesive system are conveyed in the direction of mixing chamber and mixed together upon flowing through mixing tube;
c) optionally discarding at least the first 10 mm of tissue adhesive discharging out of mixing tube;
d) applying the tissue adhesive discharged through the continued application of pressure from mixing tube onto the wound;
e) allowing the tissue adhesive to cure.
